(19) United States
(12) Patent Application Publication
(10) Pub. No.: US 2006/0198940 A1
(43) Pub. Date: Sep. 7, 2006

(54) Method of producing particles utilizing a vibrating mesh nebulizer for coating a medical appliance, a system for producing particles, and a medical appliance

(76) Inventor: David McMorrow, Galway City (IE)

Correspondence Address: KENYON & KENYON LLP, 1500 K STREET N.W., SUITE 700, WASHINGTON, DC, 20005, US

(21) Appl. No.: 11/073,197

(22) Filed: Mar. 4, 2005

(51) Int. Cl. A01G 25/09; A61L 33/00; B05B 1/08; B05B 17/00

(52) U.S. Cl. 427/2.1; 239/1; 239/102.1; 239/102.2

(57) ABSTRACT

A method of coating a medical device is provided that includes contacting a solution with a first side of a mesh nebulizer and vibrating the mesh nebulizer. The solution including a material. The mesh nebulizer includes at least one aperture. The method also includes evaporating a solvent from the solution in a region of a second side of the mesh nebulizer that is opposite the first side. The evaporating operation forms the particle of the material. The method also include contacting the particle with the medical device. A medical appliance is provided having a coating applied by a method. A system for creating a plurality of particles is provided.

### FIELD OF THE INVENTION

The present invention relates to particle production. More particularly, the present invention relates to a method of creating particles using a vibrating mesh nebulizer, a system for creating particles, and a medical appliance produced by the method.

### BACKGROUND INFORMATION

Spray drying is conventional and is used in the manufacture of powdered foodstuffs, such as dried soups. Conventional spray-drying utilizes a two (2) fluid spray atomiser to produce droplets of solution containing dissolved solids. The atomiser delivers an atomised suspension into a stream of heated gas. The heated gas causes the solvents to evaporate from the atomised droplets, causing the dissolved solids to precipitate and produce powder particles. A series of centrifugal cyclone separators may be used to separate dried particles from moist particles in the system.

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Metal stents may be coated with a polymeric coating that may contain a dissolved and/or suspended bioactive agent. The bioactive agent and the polymeric coating may be dissolved in a solvent mix and spray coated onto the stents. The solvent may then evaporate to leave a dry coating on the stent.

Conventional spray-coating technology may require nitrogen gas in order to produce a spray plume. This may result in a very high velocity spray plume. Because of the high velocity spray plume, long distances between a spray nozzle and a stent may be used in order to deliver a good coating finish. This may result in poor material efficiency, sometimes on the order of 1%. Furthermore the use of nitrogen gas may increase manufacturing costs.

Webbing may be a problem with two-fluid gas atomisers, particularly when coating large vessel coronary stents.

In the manufacture of a drug eluting stent, there are a number of challenges. Goals in the manufacture of coating stents include precise coating weight and complete encapsulation of stent struts, with minimal webbing between struts. Additionally, a stent may preferably be coated with a uniform coating on the inside and the outside of the stent and may be required to meet a product specification for kinetic drug release (KDR).

Medical appliances may be coated using spray technology. This may entail the use of a two-fluid atomiser, or spray nozzle. The atomiser may be supplied with coating solution and nitrogen gas. The nozzle may be configured so that the coating solution forms a thin film on the pre-filming face of the nozzle, and droplets may then be sheared off the film by the flow of atomising gas.

Spray coating may have a number of limitations. In a spray coating operation, droplet size and droplet velocity may be inextricably linked. It may not be possible to control either of these factors without impacting the other. Additionally, droplet size may only be controlled within a relatively large window due to the gas atomization process. Atomization energy is provided by the nitrogen gas stream. This may result in a very high velocity with a correspondingly high energy spray plume, which is a significant contributor to difficulty in fixturing stents during the coating process.

Droplet size may be a critical factor in controlling kinetic drug release. Precise control of droplet size may be important in order to develop a high degree of control of KDR.

Furthermore, it has been shown that the high velocity spray plume produced by two-fluid atomisers may cause stents to get blown out of alignment on the stent coating fixtures. This has led to difficulty in controlling coat weight, and has led to coating bare spots due to uncontrolled interaction between a stent and a coating fixture. One approach in response to this has been to significantly increase the nozzle-to-stent distance. While this reduces the movement of the stent on the coating fixture, it may result in low coating material efficiencies, perhaps on the order of 1%. A further disadvantage of two-fluid atomisers is that many of the droplets may bounce off the object to be coated, which may further limit the material efficiency. The coating of flexible, self-expanding stents and/or longer stents may create a further difficulty whereby the stent is moved, flexed and/or bent on a fixture during coating. There is therefore a need for reducing coating defects in medical appliances.

Each of the references cited herein is incorporated by reference herein for background information.

### BRIEF DESCRIPTION OF THE DRAWINGS

FIG. 1 is a schematic diagram of an exemplary system according to the present invention.

FIG. 2 is a zoomed-in view of an exemplary embodiment of a nebulizer.

FIG. 3 illustrates an exemplary embodiment of the present invention including a coating chamber.

FIG. 4 is a schematic diagram of an exemplary embodiment of a nebulizer.

FIG. 5 is another schematic diagram of another exemplary embodiment of a nebulizer.

FIG. 6 is a flowchart illustrating an exemplary method according to the present invention.

FIG. 7 is a schematic diagram of an exemplary embodiment of a nebulizer for producing particles including a gas source.

FIG. 8 is another schematic diagram of another exemplary embodiment of a nebulizer for producing particles and another nebulizer for coating a stent with a suspension.

### DETAILED DESCRIPTION

A method of coating a medical device is provided that includes contacting a solution with a first side of a mesh nebulizer and vibrating the mesh nebulizer. The solution including a material. The mesh nebulizer includes at least one aperture. The method also includes evaporating a solvent from the solution in a region of a second side of the mesh nebulizer that is opposite the first side. The evaporating operation forms the particle of the material. The method also include contacting the particle with the medical device.

The mesh nebulizer may form at least one droplet of the solution including the material.

The evaporating operation may include flowing a gas in the region of the second side. The flowing gas may be heated.

The method may include transporting the particle to a coating suspension. The particle may be insoluble in the coating suspension. The coating suspension may include a polymer.

The method may include contacting the coating suspension with a first side of a further mesh nebulizer and vibrating the further mesh nebulizer. The further mesh nebulizer may include at least one further aperture. The method may include arranging a medical appliance in a region of a second side of the further mesh nebulizer. The second side of the further mesh nebulizer may be opposite the first side of the further mesh nebulizer.

The method may include adding a surfactant to the solution including the material prior to the evaporating operation. The surfactant may prevent the particle from agglomerating with other particles. The method may include adding a surfactant to the particle after the evaporating operation, the surfactant preventing the particles from agglomerating with other particles.

The solution including the material may include at least one of toluene and tetrahydrofuran. The material may include paclitaxel.

The method may include selecting a frequency and/or an amplitude of the vibration of the mesh nebulizer.

The method may include determining a desired size of the particle, determining a size of the at least one aperture, determining a droplet size of the droplet, and determining a desired concentration of the material in the solution.

A medical appliance is provided having a coating applied by a method. The method includes contacting a solution with a first side of a mesh nebulizer and vibrating the mesh nebulizer. The solution includes a material and the mesh nebulizer includes at least one aperture. The method further includes evaporating a solvent from the solution in a region of a second side of the mesh nebulizer. The second side is opposite the first side. The evaporating operation forms a particle of the material. The method further includes transporting the particle to a coating suspension and contacting the coating suspension with a first side of a further mesh nebulizer. The further mesh nebulizer includes at least one further aperture. The method further includes vibrating the further mesh nebulizer and arranging the medical appliance in a region of a second side of the further mesh nebulizer. The second side of the further nebulizer is opposite the first side of the further nebulizer.

A system for creating a plurality of particles is provided. The system includes a solution source adapted to provide a solution including a material and a first mesh nebulizer adapted to form droplets of the solution. The system also includes a gas source adapted evaporate the solution and form the particles of the material

The system may include a polymer source adapted to provide a polymer. The material may be insoluble in the polymer. The system may include a second mesh nebulizer adapted to form droplets of the polymer having the particles in suspension.

The system may include an arrangement for holding an object. The suspension may coat the object.

An exemplary embodiment of the present invention proposes a method of generating nano-particles. In particular, the present invention proposes the use of nebuliser technology, in combination with a spray-drying process, to produce precisely sized particles of paclitaxel. These particles may then be sprayed in suspension form onto a stent. Precise particle sizing of active pharmaceutical ingredients may enable precise control of a kinetic drug release (KDR) rate.

A stent may be coated in a solution of a polymer drug carrier and a drug. The polymeric carrier and the drug may be dissolved in solvents and spray coated onto the stent. During the spray coating process, the drug may precipitate into particles, which may become dispersed throughout the polymeric drug carrier on the coated stent. The size and distribution of these drug particles may be an important factor impacting the KDR rate of the coated product.

To accurately control the drug particle size on the coated stent, it may be advantageous if the drug particle sizing could be controlled independently of the spray coating process.

A nanoparticle drug suspension may be utilized to coat stents. This approach may involve the coating of stents using a suspension, in which the particle size is pre-determined independently of spray coating parameters. Nano-milled particles may be used in suspensions for coating objects, including stents.

Nebuliser technology may be utilized to coat stents. The use of nebulisers may offer a number of potential advantages, including, but not limited to, highly precise droplet sizing. Nebulisers may be used to coat stents using a SIBs/paclitaxel solution. Nebuliser technology may be utilized to produce pre-sized paclitaxel particles, which can then be utilized for the coating of stents using a suspension approach.

The nebulizer particle production process may be utilized to produce paclitaxel particles. The paclitaxel may be dissolved in a suitable solvent (such as THF or toluene), and then sprayed. The solvent may then be evaporated from the resulting droplets, thus producing particles of precipitated paclitaxel. While this approach will certainly provide particles, the disadvantage is that the actual size, and size distribution of the particles may be large, due to the wide size range of droplets produced by the two fluid atomiser.

The nebuliser technology may be used to produce droplets of paclitaxel solution for spray drying. This approach may provide a narrow distribution of droplet sizes produced by the nebuliser, and the resulting particles may also have a narrow distribution. The narrow distribution of particle size may enable the particles to be used for the manufacture of drug eluting stents with a predictable KDR performance.

A typical droplet size produced by a nebuliser may be of the order of 5 microns in diameter, or 5000 nanometers. As an example, if the atomised suspension contains 1% paclitaxel and 99% solvent, the resulting particles may be correspondingly smaller. The size of the resulting particles may be calculated using the following steps: 1) calculate a volume of a droplet having a specified diameter (for instance, 5 microns) using the formula for the volume of a sphere (=4/3·p·r cubed); 2) divide the resulting volume of the droplet by the solids/solvent ratio, in this case 1:100 (however, this may be any appropriate ratio, for instance 1:1000, or any number depending on the required particle size) and the resulting number is the volume of the particle; and 3) calculate a diameter of the resultant particle using the formula for the volume of a sphere presented above (=4/3·p·r cubed), solving for r, and then multiplying by two (2) to obtain the diameter.

The actual particle size may be tailored by appropriate adjustment of the nebuliser control parameters, as well as by adjusting the dissolved solid to solvent ratio in the paclitaxel/solvent (or alternative material) solution. Typical paclitaxel particle size in the Taxus™ product may be on the order of 15 microns, but may have a wide size distribution.

In order to prevent the particles from agglomerating, a surfactant may be added to the paclitaxel/solvent solution prior to introducing the solution to the spray drying system. Furthermore, the equipment may be configured to capture the paclitaxel nanoparticles in suspension rather than in dry form, which may aid in the handling of the particles. This may be achieved by setting up the nebuliser so that the particles are deposited on the surface of a circulating liquid. The particles may be insoluble in the circulating liquid to prevent dissolution. The circulating liquid may be a polymer coating which may hold the particles in suspension and which may subsequently be sprayed or nebulized onto an object or medical appliance.

Alternate materials and/or coatings are possible, and in particular, the exemplary method may be used with any appropriate active pharmaceutical ingredient. Additionally, the exemplary method may be used to manufacture embolic particles for treatment of tumors. Alternate applications may include oncology, and in particular tumor treatment using embolic particles.

Nebulisers are medical devices used to vaporise medications for inhalation, specifically to convert liquid drugs into fine droplets for inhalation. Small, controllable droplet size, with typical size ranges in the order 1 to 5 microns, may be achievable with a nebulizer. A low energy droplet cloud may be desirable and therefore converting a solution into small droplets without imparting high velocities to the droplets may be desired. Additionally precise control of a delivered drug volume may be desirable.

A component of some nebuliser designs is a convex mesh which may have numerous, precisely-sized holes. The drug to be administered may be placed in the concave side of the mesh, and the mesh may be vibrated at high frequency using a piezoelectric drive. This may result in the drug being converted into a cloud of small droplets, which may be delivered on the lower (convex) side of the mesh.

Use of nebulisers instead of two-fluid atomisers may offer several advantages in coating drug eluting stents, or any other medical device. Extremely precise droplet size may be possible with a nebulizer. Precise droplet size control may be advantageous since it has been demonstrated that droplet size correlates directly to kinetic drug release (KDR). Precise control of KDR may be achievable with precise control of droplet size. Additionally, droplet size may be programmable. In particular, geometric changes may be made to the nebuliser to provide a specific desired droplet size. Additionally, droplet size may be controlled independently of droplet velocity. Due to the low velocity of the plume coupled with fine droplet size, very small stent features may be coated without webbing. No atomisation gas may be required.

Use of this method of atomisation may offer several advantages. The size of the droplets may be extremely precise because it may be determined by the size of the holes in the mesh (which may be tailor-made to suit the application). This may contribute to precise control of KDR and an ability to coat complex geometries with small feature dimensions. Due to the absence of atomisation gas, the droplets may fall away from the mesh under the force of gravity at low velocity. The volume of liquid atomised, and the droplet velocity, can also be precisely controlled by adjusting the frequency and amplitude of the mesh vibration. Furthermore, the number of holes in the mesh and their layout on the mesh can be tailored. This could enable greatly increased coating material efficiency, as the atomised cloud could be sized to suit the stent being coated. Furthermore, fixturing of stents during the coating process can be greatly simplified, as there is no longer a need to hold the stent securely to prevent it getting blown away by the atomisation gas. This may be particularly important for future generation stents which may be longer and more easily damaged during handling.

An electrostatic system may be integrated with the nebuliser. This may enable higher material efficiency while retaining precise droplet size. No atomisation gas may be required in the exemplary method, and consequently stent fixturing may be greatly simplified. Therefore, the coating process may be well controlled. An electrostatic system may be accomplished by attaching a power source to the nebuliser mesh and providing a grounding contact to the stent. This may deliver higher material efficiency.

Since nebulizers may not require a propellant gas, there may be fewer factors controlling the aerosol properties. However, the aerosol plume may require a gas current to entrain the plume so that it flows in the direction of the stent. This gas flow may be directed and accelerated towards the stent by means of a venturi type baffle arrangement.

A nebuliser may be configured in a number of ways to facilitate stent coating. In particular, mesh hole size, location and quantity may be altered. Vibration frequency and amplitude may also be tailored. Materials may be changed to facilitate use with solvent-based coatings.

The stent may be rotated and/or moved axially, or alternatively may remain fixed, depending on the size of the atomised cloud. Stent fixturing may be accomplished by supporting the stent on a pair of wires, possibly without the need to pass a wire through the center of the stent. This may accelerate the stent fixturing process, and substantially improve the quality of the stent coating, particlarly on the stent internal surface. Furthermore, this method may enable the coating of more delicate stents with increasingly complex feature details.

The design of the nebuliser may facilitate the delivery of more than one fluid to the rear surface of the mesh, thus enabling coat mixing at the point of application. This may offer benefits where short shelf-life materials are used in coating, or in the use of coating materials which are not suitable for long-term storage when pre-mixed. This approach may also be used to alter coat composition during the application of coating, thus enabling creation of products where KDR or coat composition can be altered for different areas of the product being coated.

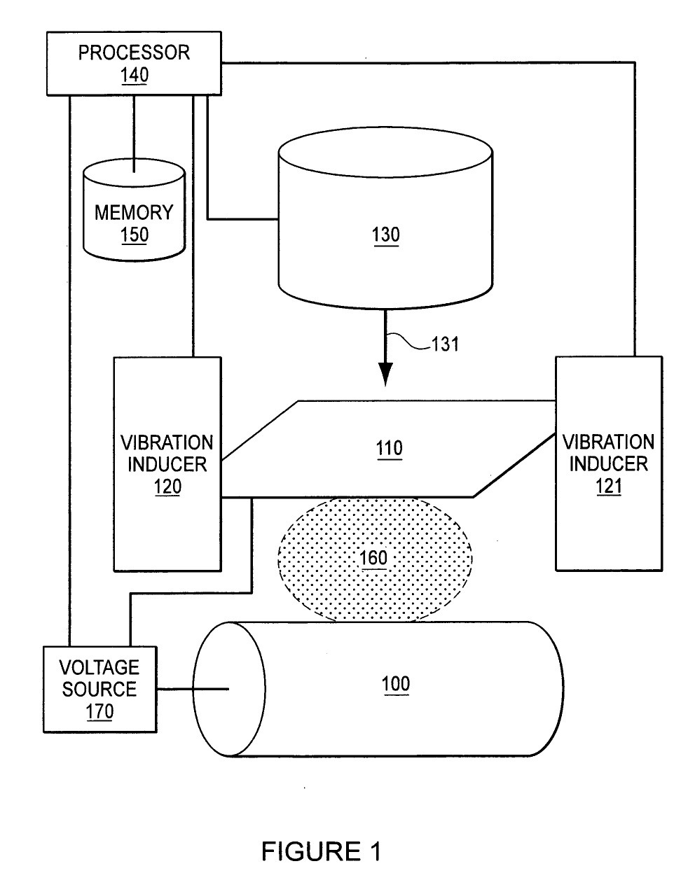

FIG. 1 is a schematic diagram of an exemplary system according to the present invention. Stent 100 is shown positioned below nebulizer mesh 110. Nebulizer mesh 110 is positioned between vibration inducers 120, 121. Alternatively, there may be more or fewer vibration inducers 120, 121. Vibration inducers 120, 121 may induce vibration in a direction parallel and/or perpendicular to nebulizer mesh 110, and may induce a complex vibration. Nebulizer mesh 110 includes one or more pores that may be between about 0.1 μm and about 200 μm, may be between about 3 μm and about 20 μm, and may be about 10 μm. The pores in nebulizer mesh 110 may be of uniform size or may be variably sized. Additionally, the pores in nebulizer mesh 110 may be frustoconical, vortex-shaped, and/or any other appropriate shape. Coating source 130 provides a coating material in the direction of arrow 131 to nebulizer mesh 110. After passing through the pores of nebulizer mesh 110, the coating material may form plume 160, which may consist of droplets. Droplets having a diameter of about 5 microns may be produced by a pore size of 3 microns in nebulizer mesh 110. The droplets in plume 160 may have a very narrow size distribution, and therefore may produce a uniform coating on stent 100. Processor 140 coupled to memory 150 may contain and/or execute instructions for operating coating source 130, vibration inducers 120, 121, and/or voltage source 170. Voltage source 170 may be connected to stent 100 and/or nebulizer mesh 110 and may impart an electric potential that provides a charge to the droplets in plume 160 that is opposite to the charge on stent 100. Plume 160 may be directed to coat stent 100 by gravity, by an additional gas source, and/or by an electrostatic potential.

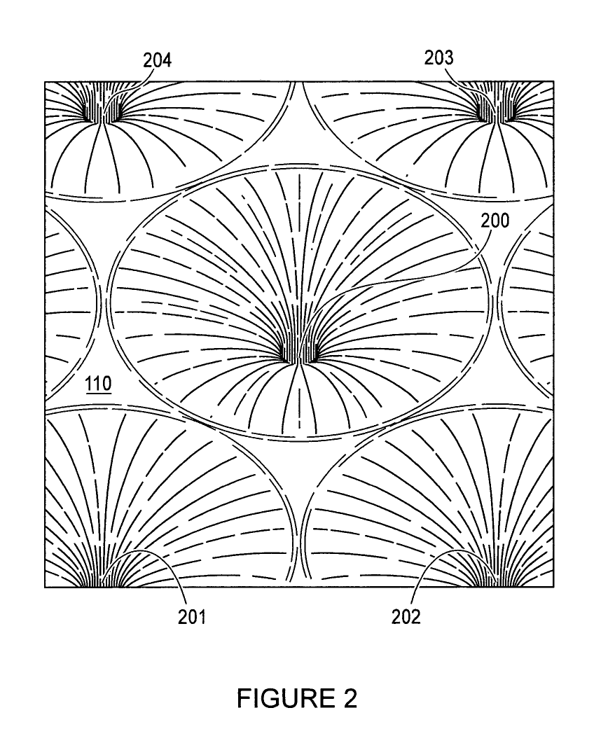

FIG. 2 is a zoomed-in view of an exemplary embodiment of nebulizer mesh 110. Nebulizer mesh 110 includes pores 200, 201, 202, 203, 204, which in this exemplary embodiment are vortex-shaped. Alternatively, pores 200, 201, 202, 203, 204 of nebulizer mesh 110 may be frusto-conical or any other appropriate shape.

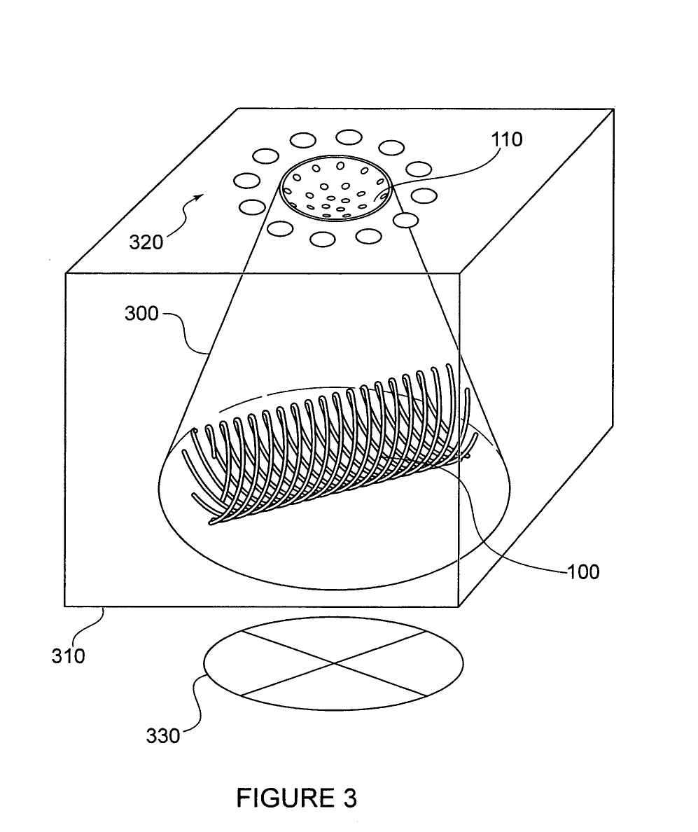

FIG. 3 illustrates an exemplary embodiment of the present invention including coating chamber 310. Nebulizer mesh 110 is situated at an upper portion of coating chamber 310. Coating chamber 310 encloses stent 100. Coating chamber 310 includes gas intakes 320, which may allow a gas to enter coating chamber 310. Gas intakes 320 may also provide a flow of gas under pressure to coating chamber 320. Gas exhaust 330 may remove gas and or excess material (for instance, coating material that has not adhered to stent 100) from coating chamber 320. Alternatively, coating chamber 310 may be airtight and/or evacuated, or may enclose an inert gas. When a coating material is arranged on mesh nebulizer 110, and mesh nebulizer 110 is vibrated, cone plume 300 of coating material in coating chamber 310 may be formed. Stent 100 may be arranged in cone plume 300. Cone plume 300 may include droplets that settle on stent 100 due to gravity, or may be assisted in moving toward stent 100 by a gas flowing from gas intakes 320 to gas exhaust 330.

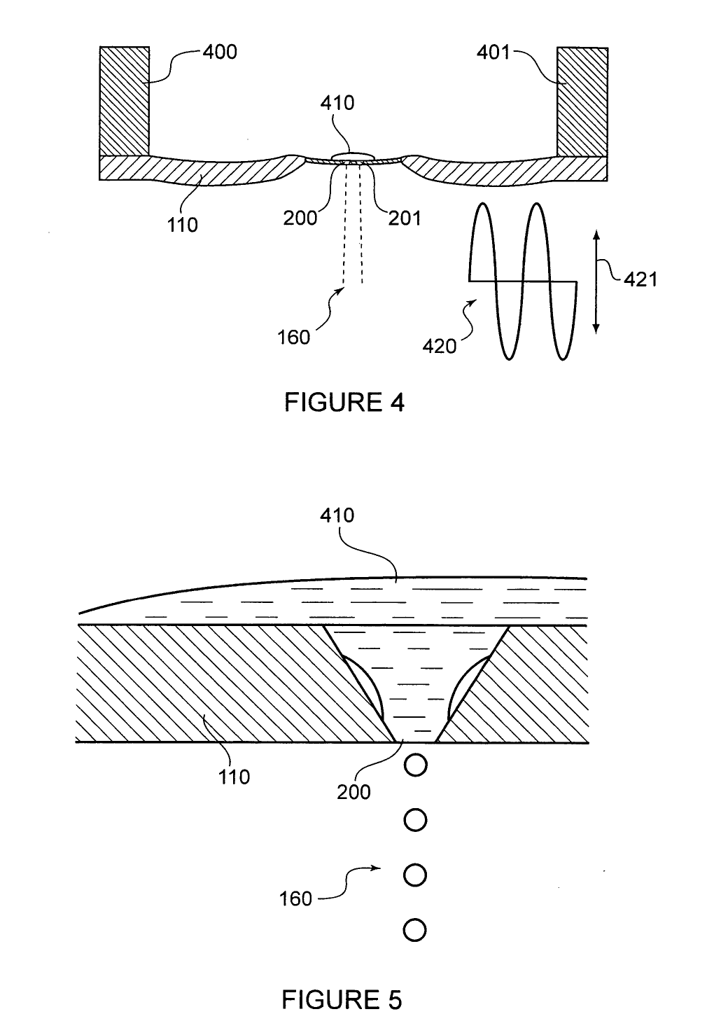

FIG. 4 is a schematic diagram of an exemplary embodiment of mesh nebulizer 110. Mesh nebulizer 110 includes pores 200, 201 and lateral barriers 400, 401. Alternatively, there may be more or fewer pores 200, 201, and/or more or fewer lateral barriers 400, 401. Coating material 410 is situated on a top side of mesh nebulizer 110, and is situated in a vicinity of pores 200, 201. Lateral barriers 400, 401 and/or another element may impart a vibration to mesh nebulizer. The vibration may correspond to sinusoid 420, and may consist of a vibration in a direction of double arrow 421. Alternatively or additionally, a lateral vibration in a plane of nebulizer mesh 110 may be induced. The vibration of nebulizer mesh 110 may induce coating material 410 to pass through pores 200, 201 to create plume 160.

FIG. 5 is another schematic diagram of another exemplary embodiment of nebulizer mesh 110 showing a zoomed in view of pore 200. Pore 200 is frustoconical, though alternative shapes may be possible. Coating material 410 flows through pore 200 when nebulizer mesh 110 is vibrated to form plume 160, which may be composed of droplets of a small diameter. The droplets of plume 160 may have a narrow size distribution, and may be between about 0.1 μm and about 200 μm, or may be between about 3 μm and about 20 μm. In one exemplary embodiment, pore 200 may be about 3 microns in diameter and the droplets in plume 160 may be about 5 microns in diameter.

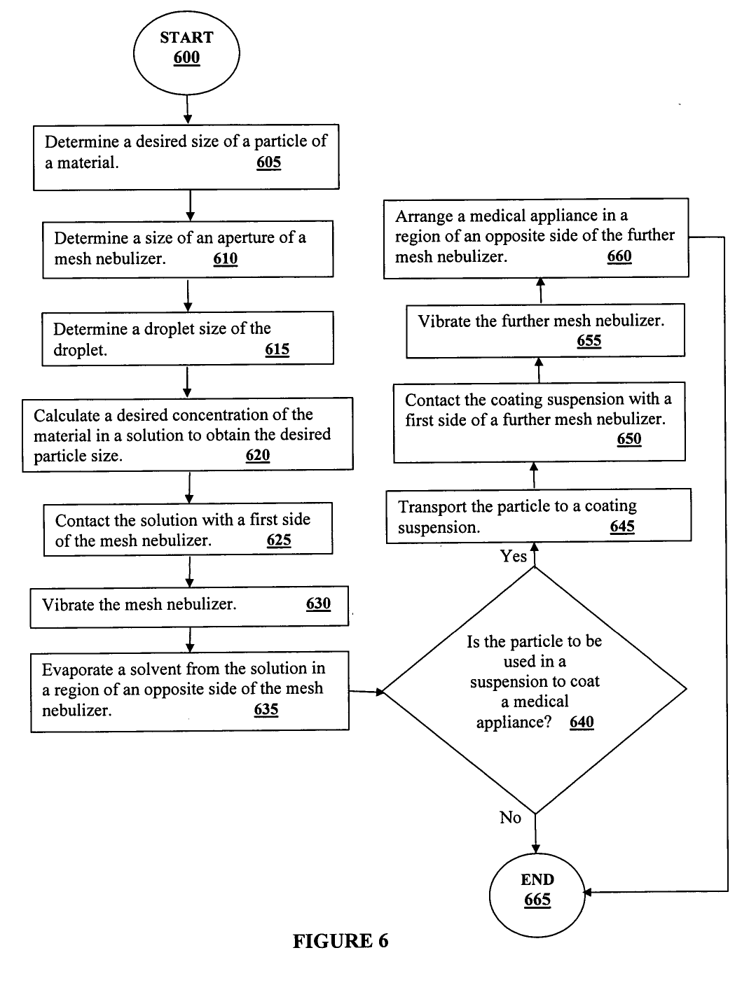

FIG. 6 is a flowchart illustrating an exemplary method according to the present invention. The flow in FIG. 6 starts in start circle 600 and proceeds to action 605, which indicates to determine a desired size of a particle of a material. From action 605, the flow proceeds to decision 610, which indicates to determine a size of an aperture of a mesh nebulizer. From action 610, the flow proceeds to action 615, which indicates to determine a droplet size of the droplet. From action 615, the flow proceeds to action 620, which indicates to calculate a desired concentration of the material in a solution to obtain the desired particle size. From action 620, the flow proceeds to action 625, which indicates to contact the solution with a first side of a mesh nebulizer. From action 625, the flow proceeds to action 630, which indicates to vibrate the mesh nebulizer. From action 630, the flow proceeds to action 635, which indicates to evaporate a solvent from the solution in a region of an opposite side of the mesh nebulizer. From action 635, the flow proceeds to decision 640, which asks whether the particle is to be used in a suspension to coat a medical appliance. If the response to decision 640 is affirmative, the flow proceeds to action 645, which indicates to transport the particle to a coating suspension. From action 645, the flow proceeds to action 650, which indicates to contact the coating suspension with a first side of a further mesh nebulizer. From action 650, the flow proceeds to action 655, which indicates to vibrate the further mesh nebulizer. From action 655, the flow proceeds to action 660, which indicates to arrange a medical appliance in a region of an opposite side of the further mesh nebulizer. From action 660, the flow proceeds to end circle 665. If the response to decision 640 is negative, the flow proceeds to end circle 665.

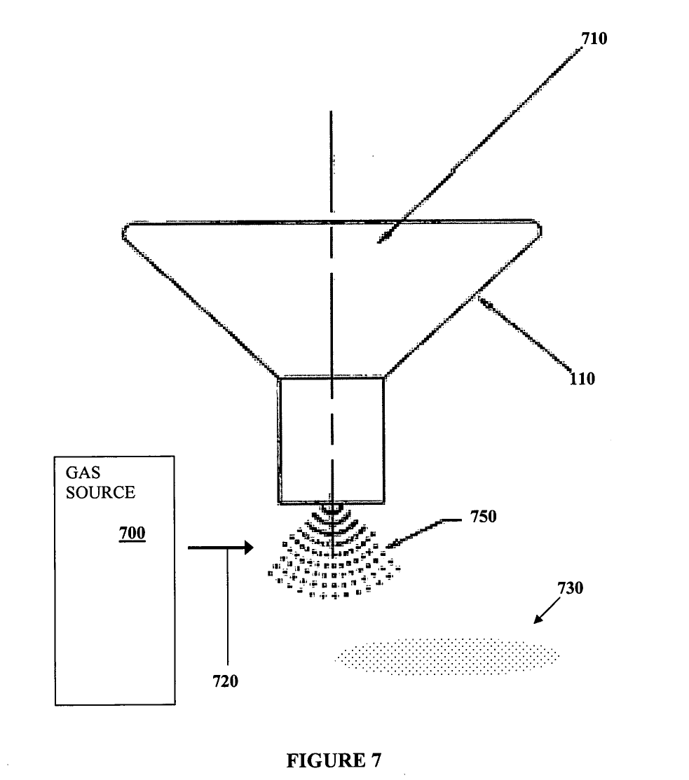

FIG. 7 is a schematic diagram of an exemplary embodiment of nebulizer 110 including gas source 700. Solution 710 contacts a first side of nebulizer 110, which operates to form droplets 750 of solution 710. Solution 710 may be a bioactive agent in solution, and may be paclitaxel dissolved in toluene or tetrahydrofuran. The ratio of bioactive agent to solvent in solution 710 may determine the size of particles 730 which are produced by the system. Additionally, the size of the aperture or apertures in nebulizer 110 may also determine the size of particles 730 by determining the size of droplets 750. Droplets 750 emerge from a second size of nebulizer 110 when nebulizer 110 is vibrated. Gas source 700 may be directed at droplets 750 as they emerge from nebulizer 110 and may operate to dry droplets 750 by directing gas at droplets 750 in the direction of arrow 720. Gas source 700 may also heat the gas flowing in the direction of arrow 720 to promote the evaporation of the solvent in solution 710. As the solvent in solution 710 evaporates, a single particle 730 may precipitate out of each droplet 750. A surfactant may be introduced to solution 710 to prevent the agglomeration of particles 730. Alternatively or additionally, a surfactant may be arranged on particles 730 before or during their precipitation from droplets 750. In particular, a surfactant may be introduced via gas source 700. Nebulizer 110 may be provided with only one aperture, or only widely spaced apertures, in order to prevent and/or discourage the agglomeration of particles 730. Particles 730 may have a narrow size distribution due to the fact that droplets 750 may have a narrow size distribution and the ratio of solvent to material in solution 710 may be uniform. Particles 730 may thereafter be used in any context requiring particles of a uniform size.

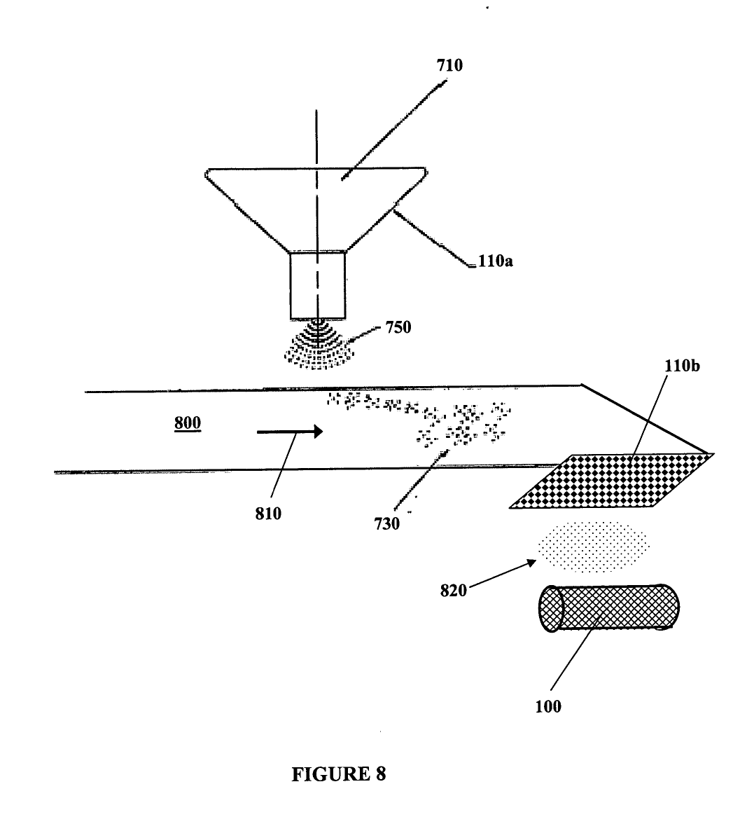

FIG. 8 is another schematic diagram of another exemplary embodiment of nebulizer 110 for producing particles and another nebulizer 110a for coating stent 100 with particles 730 in suspension 800. Solution 710 contacts a first side of nebulizer 110a, which operates to form droplets 750 of solution 710. Solution 710 may be a bioactive agent in solution, and may be paclitaxel dissolved in toluene or tetrahydrofuran. Droplets 750 emerge from a second size of nebulizer 110a when nebulizer 110a is vibrated. The solvent in solution 710 is allowed or encouraged to evaporate, either by the passage of time, or by introduction of a heated gas as shown in FIG. 7. A single particle 730 may precipitate out of each droplet 750. Particles 730 may deposit in liquid 800, which may be a polymer. The material of particle 730 may be insoluble in liquid 800 in order to maintain the particle structure in suspension. A surfactant may be introduced via liquid 800 to prevent the agglomeration of particles 730. Liquid 800 including particles 730 may be directed toward a first side of another nebulizer 110b which may vibrate and create suspension droplets 820, which may be droplets having a narrow size distribution and including particles 730 having a narrow size distribution. Suspension droplets 820 may include a polymer (for instance SIBs), and may include a bioactive agent (for instance, paclitaxel). Suspension droplets 820 may be deposited on stent 100, thereby coating stent 100 with a polymer including a bioactive agent.

As used herein, the term “therapeutic agent” includes one or more “therapeutic agents” or “drugs”. The terms “therapeutic agents”, “active substance” and “drugs” are used interchangeably herein and include pharmaceutically active compounds, nucleic acids with and without carrier vectors such as lipids, compacting agents (such as histones), virus (such as adenovirus, andenoassociated virus, retrovirus, lentivirus and α-virus), polymers, hyaluronic acid, proteins, cells and the like, with or without targeting sequences.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin-) cells including Lin-CD34−, Lin-CD34+, Lin-cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

What is claimed is:

1. A method of coating a medical device, comprising:
contacting a solution with a first side of a mesh nebulizer, the solution including a material, the mesh nebulizer comprising at least one aperture;
vibrating the mesh nebulizer;
evaporating a solvent from the solution in a region of a second side of the mesh nebulizer to form a particle of the material, the second side opposite the first side; and
contacting the particle with the medical device.

2. The method of claim 1, wherein the mesh nebulizer forms at least one droplet of the solution including the material.

3. The method of claim 2, wherein the evaporating operation includes flowing a gas in the region of the second side.

4. The method of claim 3, wherein the flowing gas is heated.

5. The method of claim 1, further comprising transporting the particle to a coating suspension.

6. The method of claim 5, wherein the particle is insoluble in the coating suspension.

7. The method of claim 5, wherein the coating suspension comprises a polymer.

8. The method of claim 5, further comprising:
contacting the coating suspension with a first side of a further mesh nebulizer, the further mesh nebulizer comprising at least one further aperture;
vibrating the further mesh nebulizer; and
arranging the medical appliance in a region of a second side of the further mesh nebulizer, the second side of the further mesh nebulizer opposite the first side of the further mesh nebulizer.

9. The method of claim 1, further comprising adding a surfactant to the solution including the material prior to the evaporating operation, the surfactant preventing the particle from agglomerating with other particles.

10. The method of claim 1, further comprising adding a surfactant to the particle after the evaporating operation, the surfactant preventing the particles from agglomerating with other particles.

11. The method of claim 1, wherein the solution including the material comprises at least one of toluene and tetrahydrofuran.

12. The method of claim 1, wherein the material comprises paclitaxel.

13. The method of claim 1, further comprising selecting at least one of a frequency and an amplitude of the vibration of the mesh nebulizer.

14. The method of claim 1, further comprising:
determining a desired size of the particle;
determining a size of the at least one aperture;
determining a droplet size of the droplet; and
determining a desired concentration of the material in the solution.

15. A medical appliance having a coating applied by a method, the method comprising:
contacting a solution with a first side of a mesh nebulizer, the solution including a material, the mesh nebulizer comprising at least one aperture;
vibrating the mesh nebulizer;
evaporating a solvent from the solution in a region of a second side of the mesh nebulizer, the second side opposite the first side, the evaporating operation forming a particle of the material;
transporting the particle to a coating suspension;
contacting the coating suspension with a first side of a further mesh nebulizer, the further mesh nebulizer comprising at least one further aperture;
vibrating the further mesh nebulizer; and
arranging the medical appliance in a region of a second side of the further mesh nebulizer, the second side of the further nebulizer opposite the first side of the further nebulizer.

16. A system for creating a plurality of particles, comprising:
a solution source adapted to provide a solution including a material;
a first mesh nebulizer adapted to form droplets of the solution; and
a gas source adapted evaporate the solution and form the particles of the material.

17. The system of claim 16, a polymer source adapted to provide a polymer, the material being insoluble in the polymer.

18. The system of claim 16, further comprising a second mesh nebulizer adapted to form droplets of the polymer having the particles in suspension.

19. The system of claim 16, further comprising an arrangement for holding an object, the suspension coating the object.
